SEQUENCE LISTING (4 sequences)

SEQ ID NO: 1 - protein, 4 aa, Homo sapiens
  misc_feature (2)..(3): Xaa can be any naturally occurring amino acid
YXXL

SEQ ID NO: 2 - protein, 6 aa, Homo sapiens
AMPGAE

SEQ ID NO: 3 - protein, 10 aa, Homo sapiens
GVAMPGAEDD

SEQ ID NO: 4 - protein, 8 aa, Homo sapiens
  misc_feature (3)..(4): Xaa can be any naturally occurring amino acid
EDXXVTPG

(19) United States
(12) Patent Application Publication
(10) Pub. No.: US 2019/0209680 A1
(43) Pub. Date: Jul. 11, 2019

(54) TREATMENT OF ACUTE LIVER FAILURE

(71) Applicant: The University of Birmingham, Birmingham (GB)

(72) Inventors: Abhishek Chauhan, Birmingham, West Midlands (GB); Patricia Frances Lalor, Birmingham, West Midlands (GB); Stephen Paul Watson, Birmingham, West Midlands (GB)

(21) Appl. No.: 16/331,013

(22) PCT Filed: Sep. 6, 2016
(86) PCT No.: PCT/GB2016/052742

§ 371 Date: Mar. 6, 2019

(51) Int. Cl. A61K 39/395; C07K 16/28; A61K 31/197; A61K 31/565; A61P 1/16; A61K 45/06

(52) U.S. Cl. CPC A61K 39/3955; C07K 16/2851; A61K 31/197; A61K 2039/505; A61P 1/16; A61K 45/06; C07K 2317/24; A61K 31/565

(57) ABSTRACT

The present invention relates to the treatment or prophylaxis of acute liv-failure. More particularly, the invention relates to use of an agent that modulates the podoplanin pathway, such as by inhibiting an interaction of podoplanin with CLEC-2 or inhibiting the activity of Src and/or Syk family kinases for the treatment or prophylaxis of acute liver failure, as well as a method for determining the efficacy of treatment of acute liver failure.

### FIELD OF THE INVENTION

The present invention relates to the treatment or prophylaxis of acute liver failure. More particularly, the invention relates to use of an agent that modulates the podoplanin pathway, such as by inhibiting an interaction of podoplanin with CLEC-2 or inhibiting the activity of Src and/or Syk family kinases for the treatment or prophylaxis of acute liver failure, as well as a method for determining the efficacy of treatment of acute liver failure.

### BACKGROUND TO THE INVENTION

Acute liver failure is a life-threatening critical illness that most often occurs in patients who do not have pre-existing liver disease. With the incidence of acute liver failure rising, its healthcare burden and costs are expected to continue to rise.

Various causes of acute liver failure have been identified. These include viral infections (for example hepatitis A, B and E infection, herpes simplex virus, cytomegalovirus, Epstein-Barr virus and parvoviruses), drug-induced liver injury (for example acetaminophen-induced), alcohol-induced liver injury, autoimmune disease, heatstroke or toxin-induced liver failure. Other causes include hypoxic hepatitis as a result of primary cardiac, circulatory, or respiratory failure, or acute liver failure during pregnancy.

Many patients with acute liver failure die or require transplantation. Alcoholic hepatitis in particular has a 28 day mortality of up to 35%. Despite the high mortality rates, treatment options remain limited. Other than transplantation, treatment options are limited to corticosteroids or NAC (N-acetyl cysteine). Unfortunately, not all patients respond to treatment. Some patients are also too critically ill to be suitable for transplantation. For the patients who undergo transplantation and ultimately recover, they will then require life-long immunosuppressive treatment to prevent rejection of the transplant. This is very costly.

The clinical decision-making process for the treatment of acute liver failure is complex. Evaluation of the severity of the liver failure and the resulting selection of treatment is crucial for preventing patient mortality. In some instances, transplantation may not be required but is carried out, due to the acute onset of failure and the short time frame in which to make a clinical decision. This leads to the unnecessary wastage of donor organs.

Platelets are fundamental players in liver pathobiology, driving inflammation, fibrosis, cancer and even aiding regeneration. CLEC-2 (C-type lectin-like receptor 2) is a type II transmembrane protein which is expressed on platelets. Platelet-based CLEC-2 mediates platelet activation on meeting its ligand Podoplanin, a type I transmembrane O-glycoprotein. Podoplanin comprises an extracellular domain with abundant Ser and Thr residues, a single transmembrane protein and a short cytoplasmic tail.

The specific molecular basis of platelet activation in the context of liver inflammation and thus failure remains elusive.

The present invention has been devised with these issues in mind.

### DESCRIPTION

Broadly speaking, the present invention is based upon modulation of the podoplanin pathway, such as through the inhibition of the interaction of podoplanin with CLEC-2. In the context of the present invention, the podoplanin pathway will be understood to refer to an interaction of podoplanin with CLEC-2 and certain downstream targets of the interaction. As the skilled person will appreciate, podoplanin has a single transmembrane region and short cytoplasmic tail that interacts with members of the ERM family of proteins to link podoplanin to the actin cytoskeleton. The interaction of podoplanin with CLEC-2 results in phosphorylation of tyrosine residues in an YXXL motif in the intracellular ITAM domain of CLEC-2 and permits CLEC-2 to interact with tyrosine kinases such as Src and Syk. This leads to activation of other downstream partners such as SLP-76 and PLCy and causes platelet activation and aggregation. Thus, the pathway may be inhibited by inhibition of the interaction of podoplanin with CLEC-2, or by inhibition of the activity of certain downstream targets. For example, inhibition of the interaction of podoplanin with CLEC-2, or the activity of Src and/or Syk family kinases results in inhibition of the activation of other downstream partners such as SLP-76 and PLCy.

According to a first aspect of the invention there is provided an agent that inhibits an interaction of podoplanin with CLEC-2, or inhibits the activity of Src and/or Syk family kinases for use in the treatment and/or prophylaxis of acute liver failure in a subject.

The present inventors have surprisingly found that acute liver failure can be prevented or treated by inhibition of the interaction of podoplanin with CLEC-2. Without wishing to be bound by theory, the inventors believe that the inhibition of the podoplanin pathway increases the secretion and/or expression of TNF-alpha and increases myeloid cell recruitment in the subject. Unexpectedly, the inventors have found that increased TNF-alpha and/or increased myeloid cell recruitment is associated with reduced liver failure and improved healing.

The “interaction of podoplanin with CLEC-2”, as used herein, will be understood as referring to the natural interaction or association between the ligand podoplanin and its receptor CLEC-2. This interaction may not require Ca²⁺. The interaction may comprise association of CLEC-2 with a PLAG (platelet aggregation-stimulating) domain of podoplanin, for example at least one of PLAG1, PLAG2, PLAG3, or PLAG4 and/or the association of podoplanin with a CTLD (C-type lectin-like domain) of CLEC-2. It will be appreciated that the interaction may comprise association between the CTLD (C-type lectin-like domain) of CLEC-2 and a disialyl-core1 in the PLAG domain of podoplanin. The interaction may occur at amino acids Glu47 and/or Asp48 in the PLAG3 domain of podoplanin. The interaction may further comprise the alpha2-6 linked sialic acid residue of podoplanin. The interaction may comprise Thr52 in the PLAG domain. Thr52 may be sialylated. In some examples the interaction comprises the PLAG2 domain of podoplanin. The interaction may comprise amino acids 38-51 of the PLAG2 domain of podoplanin. In some instances the interaction may comprise one or more glycosylation sites of podoplanin. For example, the interaction may comprise the O-glycosylation of Thr25 in the N terminus of podoplanin, as described by Kaneko et al., Mon. Anti. In Immunodiagnosis and Immunotherapy, 2015, 34(5), 310-317. It will be appreciated that the interaction may comprise association of podoplanin with the noncanonical side face of CLEC-2. The crystal structure of the interaction of podoplanin with CLEC-2 is described by Nagae et al., Structure, 2014, 22(12), 1711-1721, to which the skilled reader is directed.

As used herein, the term “PLAG domain” will be understood to refer to the EDxxVTPG segment in the extracellular domain of podoplanin.

It will be appreciated that podoplanin may interact with a CLEC-2 monomer, a CLEC-2 dimer or a CLEC-2 multimer.

By “inhibits”, as used herein, it will be understood that the agent prevents or decreases the interaction between CLEC-2 and podoplanin, or the activity of Src and/or Syk family kinases relative to normal levels (i.e. the level in the absence of the agent). Inhibition of the interaction or the activity may be partial or complete. The agent may decrease the interaction or the activity by at least 5, 10, 20, 30, 40, 50, 60, 70, 80, 90 or 100%. It will be further appreciated that the inhibition of the interaction of podoplanin with CLEC-2 or the activity of Src and/or Syk family kinases by the agent may be direct or indirect.

The agent may be capable of specifically binding to CLEC-2 or podoplanin. For example, the agent may be an antibody that specifically binds to CLEC-2 or podoplanin, thereby causing direct repression of the binding of podoplanin to CLEC-2. In some examples the agent may be capable of competitively binding to podoplanin or CLEC-2. By “competitively binding” it will be understood that the agent is capable of binding to a site on a first member, for example CLEC-2 or podoplanin, such that it prevents the binding of a second member, for example CLEC-2 or podoplanin to the first member. A suitable competitive binding agent may be a fragment of CLEC-2 or podoplanin which is capable of specifically binding to its respective partner (i.e. podoplanin or CLEC-2 respectively) and prevent or inhibit binding of a native molecule.

The agent may be capable of specifically binding to podoplanin. In some embodiments, the agent is capable of specifically binding to CLEC-2.

The agent may be capable of competitively binding to the CTLD (C-type lectin-like domain) of CLEC-2. In some embodiments the agent is capable of binding to another site of CLEC-2. The agent may be capable of competitively binding to the PLAG (platelet aggregation-stimulating) domain of podoplanin, for example at least one of PLAG1, PLAG2, PLAG3 or PLAG4. In some embodiments the agent is capable of competitively binding to the disialyl-core1 in the PLAG domain of podoplanin. In some embodiments the agent is capable of competitively binding to the PLAG2 domain of podoplanin. The agent may be capable of competitively binding to the amino acids 38-51 of the PLAG2 domain of podoplanin. In some embodiments the agent is capable of binding to one or more glycosylation sites of podoplanin. The agent may be capable of binding to the glycosylated Thr25 in the N terminus of podoplanin. In some embodiments the agent is capable of binding to another site of podoplanin.

In some embodiments, the agent is capable of specifically binding to podoplanin or CLEC-2 mRNA, thereby causing direct repression of expression of the gene into the CLEC-2 or podoplanin protein. The agent may be capable of specifically binding to podoplanin mRNA. In some embodiments the agent is capable of specifically binding to CLEC-2 mRNA.

The agent may be capable of inhibiting the activity of Src kinase. In some embodiments, the agent is capable of inhibiting the activity of Syk kinase. The agent may be capable of inhibiting the phosphorylation of Src and/or Syk kinase. The agent may be capable of specifically binding to Src kinase. In some embodiments the agent is capable of specifically binding to Syk kinase. For example, the agent may allosterically bind to Src and/or Syk kinase, resulting in a conformational change to Src and/or Syk kinase.

By “allosteric” or “allosterically”, as used herein it will be understood that the agent is capable of binding to a site of a target other than the active site of the target.

In some embodiments the agent is capable of competitively binding to the ATP-binding site or a site adjacent to the ATP-binding site of Src and/or Syk kinase. In this way the binding of ATP (adenosine triphosphate) to the ATP-binding site is inhibited and so phosphorylation of Src and/or Syk kinase is inhibited. In some examples the site adjacent to the ATP-binding site is a hydrophobic pocket. The agent may be capable of inhibiting the interaction of Src and/or Syk kinase with the Cdc37-HSp90 molecular chaperone system. By inhibiting this interaction, the Src and/or Syk kinase may be ubiquitinated and degraded.

The agent may be capable of modifying hepatic inflammation, for example hepatic necroinflammation. The term “modifying” as used herein, will be understood to refer to an increase or reduction.

In some examples the agent may be capable of reducing hepatic inflammation, for example hepatic necroinflammation. In some examples the agent may be capable of modifying hepatic levels of TNF-alpha and/or other cytokines. The agent may be capable of increasing hepatic levels of TNF-alpha and/or other cytokines. The agent may be capable of altering the proportion different macrophage sub types in the liver.

The agent may be capable of modifying neutrophil and/or myeloid cell numbers in the liver. In some examples, the agent may be capable of increasing neutrophil and/or myeloid cell numbers in the liver. In some examples, the agent may be capable of reducing alanine transaminase (ALT) levels. A reduction or increase may be relative to at the time of diagnosis or during disease.

In some embodiments the agent, is capable of modifying hepatic inflammation and hepatic TNF-alpha levels. As the skilled person will appreciate, TNF-alpha is a known pro-inflammatory cytokine. It is therefore surprising that the agent may be capable of modifying hepatic inflammation and hepatic TNF-alpha levels.

Thus, the skilled person may determine the efficacy of the agent in the treatment or prophylaxis of acute liver failure by measuring any of the level of hepatic inflammation, the number and/or type of macrophages in the liver, the number of neutrophils and/or myeloid cells in the liver, the level of ALT or the level of TNF-alpha or other cytokines. The level(s) may be measured from a sample from a subject. The sample may be a liver biopsy, blood or serum. Other suitable samples will be known to the skilled person.

By “treatment” as used herein, it will be understood that the agent reduces, alleviates or eliminates symptoms of a medical condition, disease or pathology. The term “eliminates” may be understood to refer to the complete removal of symptoms. As used herein, “alleviation” will be understood to refer to the lessening of symptoms such that the subject's quality of life is improved. For example, the alleviation of symptoms may be understood to refer to a reduction in pain and morbidity of the subject. The lessening of symptoms may be relative to at the time of diagnosis or during disease. The term “treatment” may refer to the administration of the agent after the onset of symptoms or after diagnosis.

The reduction, alleviation or elimination of symptoms may be measured using various methods. For example, the skilled medical practitioner may use a prothrombin time (PT) test, which measures how long it takes for blood to clot. A reduction of symptoms may be considered to be a reduced time period for blood to clot. This may be relative to the time taken for blood to clot at the time of diagnosis or during disease. The prothrombin time test may be used with a partial thromboplastin time (PTT) test. Other methods may include imaging tests, for example, ultrasound, to evaluate liver damage. In this context, reduced liver damage may be considered to be a reduction or alleviation of symptoms. Other methods for measuring a reduction, alleviation or elimination of symptoms may include measuring the levels of alanine transaminase (ALT) in a sample from a subject. In this context, a reduction, alleviation or elimination of symptoms may be considered to be decreased levels of ALT, relative to the ALT levels at the time of diagnosis or during disease.

Other methods for measuring the reduction, alleviation or elimination may include coagulation studies, the detection of aspartate aminotransferase (AST)/serum glutamic-oxaloacetic transaminase (SGOT), serum glutamic-pyruvic transaminase (SGPT), alkaline phosphatase (ALP) , glucose, bilirubin, ammonia, lactate, phosphate, creatinine, immunoglobulin levels, circulating antibody titres—such as circulating IgG, IgM or IgG autoantibodies or virus specific antibodies or copper and/ceruloplasmin levels in a sample from the subject. Levels to be detected may be increased or decreased relative to normal levels. Levels may be increased or decreased by at least 5, 10, 20, 30, 40, 50, 60, 70, 80, 90 or 100%. The sample may be blood, serum or urine, for example.

The skilled person will understand the term “prophylaxis” to refer to the preservation of health of a subject, for example protective and/or preventative treatment for a medical condition, disease or pathology. The term “prophylaxis” may thus refer to the reduction, alleviation or complete prevention of future symptoms.

In the context of the present invention, reduction or elimination may relate to the reduced or lessened effect of a causative factor or cause of acute liver failure. The term “prophylaxis” may thus refer to a reduction or lessening of inflammation from a causative factor or cause.

As the skilled person will appreciate, prophylaxis may be of benefit to subjects who may be at risk of developing acute liver failure. For example, prophylaxis may be of benefit to subjects who intake excess levels of toxins, alcohol, drugs or nutritional supplements. Excess levels intaken can be determined by methods known to those skilled in the art.

“Acute liver failure”, as used herein, will be understood to refer to a sudden-onset reduction or loss in liver function. The function may be reduced or lost relative to normal levels. The function may be reduced by at least 5, 10, 20, 30, 40, 50, 60, 70, 80, 90 or 100%. By “sudden onset” the skilled person will appreciate that the reduction in liver function occurs rapidly. The reduction in liver function may occur a few days, a few weeks, or a few months after exposure to a causative factor or from the onset of the disease or condition. Example diseases or conditions may include pregnancy, autoimmune disease, vascular diseases, metabolic diseases, microbial infection, drug-induced disease or alcohol exposure, for example alcoholism. Other diseases or conditions will be known to a person skilled in the art. In some embodiments the reduction in liver function occurs no more than 9 months, no more than 6 months, no more than 3 months, no more than 6 weeks, no more than 4 weeks, no more than 2 weeks, no more than 1 week, no more than 5 days, no more than 3 days, no more than 2 days or no more than 1 day after exposure to a causative factor or from the onset of the disease or condition.

Symptoms of acute liver failure may include, but may not be limited to, any of nausea, diarrhoea, fatigue, loss of appetite, jaundice, abdominal pain and/or swelling, disorientation, cerebral edema, encephalopathy, ascites, change in liver span, hematemesis, melena, hypotension, tachycardia, drowsiness or coma.

It will be appreciated that acute liver failure is distinct from chronic liver failure. Acute liver failure will be understood to refer to a sudden-onset reduction or loss in liver function, whereas chronic liver failure will be understood to refer to a gradual reduction or loss in liver function. The sudden-onset reduction or loss in liver function in acute liver failure most commonly occurs in subjects with no pre-existing liver disease. In contrast, chronic liver failure is associated with pre-existing disease, i.e. the disease is long-term. By “long-term” the skilled person will appreciate that the disease or condition is of prolonged duration, for example, of at least 12 months, at least 2 years, at least 5 years, at least 10 years, at least 20 years, at least 30 years, at least 40 years, at least 50 years, at least 60 years, at least 70 years or at least 80 years.

The distinction of acute inflammatory diseases or conditions from chronic inflammatory diseases or conditions may also lie in the concentration or number of periods by which the subject was exposed to a causative factor. Acute inflammatory diseases or conditions may be characterised by one period of exposure, or one exposure, to the causative factor, whereas chronic inflammatory diseases or conditions may be characterised by repeated exposure, for example more than one period of exposure, or persistent exposure to the causative factor.

As the skilled person will appreciate, chronic inflammatory diseases or conditions can be associated with different immune characteristics, cytokine, growth factor stimuli and/or mediators to acute inflammatory diseases or conditions. For example, chronic inflammatory diseases or conditions may be associated with the infiltration of monocyte, macrophage and/or lymphocyte subpopulations. In contrast, acute inflammatory diseases or conditions, may be associated with an infiltration and/or activation of predominantly neutrophils. Acute inflammatory disease or conditions are not commonly associated with the development of fibrosis which is a more common characteristic of chronic inflammatory diseases or conditions. Thus, acute inflammatory diseases or disorders may have distinct pro-inflammatory drivers to chronic inflammatory diseases or disorders.

Methods for diagnosing acute liver failure are known to the skilled medical practitioner. Tests for the diagnosis of acute liver failure may include coagulation studies, the detection of aspartate aminotransferase (AST)/serum glutamic-oxaloacetic transaminase (SGOT), alanine aminotransferase (ALT)/serum glutamic-pyruvic transaminase (SGPT), alkaline phosphatase (ALP), glucose, bilirubin, ammonia, lactate, phosphate, creatinine, immunoglobulin levels, circulating antibody titres or copper and/ceruloplasmin levels in a sample from the subject. The skilled practitioner may assess the levels and/or acetaminophen-product adduct levels in a sample from a subject. Other diagnosis methods may include viral serologies, the detection of autoimmune markers, electroencephalography, intracranial pressure monitoring, liver biopsy or imaging. Viral serologies may include the detection of viral surface antigen, or Immunoglobulin, for example, the detection of hepatitis A, B, C, D or E virus immunoglobulin M (IgM) or hepatitis B surface antigen (HbsAg). Liver biopsy may be percutaneous or transjugular. Imaging may include hepatic doppler ultrasonography, abdominal computed tomography (CT) scanning, magnetic resonance imaging or cranial CT scanning. Levels to be detected may be increased or decreased relative to normal levels. Levels may be increased or decreased by at least 5, 10, 20, 30, 40, 50, 60, 70, 80, 90 or 100%. The sample may be blood, serum or urine.

The diagnosis of acute liver failure may lie in the identification of the cause of the symptoms. For example, the skilled medical practitioner may diagnose acute liver failure if the subject has ingested excess toxins, excess nutritional supplements, excess alcohol or excess drugs e.g. acetaminophen. As the skilled person will appreciate, it may be important to identify the cause since certain causes necessitate rapid or immediate treatment.

Causes of acute liver failure can include viral infection, alcohol, drugs, herbal supplements, vascular diseases, for example Budd-Chiari syndrome, metabolic disease, for example Wilson's disease, cancer, autoimmune disease, heatstroke, environmental toxins, pregnancy or primary cardiac, circulatory, or respiratory failure. Viral infection may include Hepatitis A, B, C, D or E, Epstein-Barr virus, cytomegalovirus or herpes simplex virus infection. Toxins which may cause acute liver failure include the poisonous wild mushroom Amanita phalloides. The autoimmune disease may be autoimmune hepatitis. Herbal supplements which may cause acute liver failure include kava, ephedra, skullcap and pennyroyal. Drugs which have been shown to cause acute liver failure include antibiotics, nonsteroidal anti-inflammatory drugs, acetaminophen or anticonvulsants. Other causes will be known to the skilled medical practitioner.

In some embodiments the acute liver failure is selected from viral-induced, drug-induced, alcohol-induced, autoimmune-induced, heat-stroke-induced, toxin-induced, hypoxic hepatitis-induced or pregnancy-induced liver failure.

In some embodiments the acute liver failure is selected from viral induced, drug-induced, alcohol-induced, autoimmune-induced or toxin-induced liver failure. In some embodiments the acute liver failure is selected from alcohol-induced or drug-induced liver failure.

In some embodiments, the subject is a mammal. In some embodiments, the subject is human. Non-human subjects to which the invention is applicable include pets, domestic animals, wildlife and livestock, including dogs, cats, cattle, horses, sheep, goats, deer and rodents.

The subject may have been diagnosed as suffering from acute liver failure. The subject may be suspected of having acute liver failure, and/or may be displaying symptoms of acute liver failure. In some embodiments, the subject is identified as being at risk of developing acute liver failure.

The subject may have been diagnosed as suffering from hepatitis, alcoholism, drug or alcohol overdose, toxin overdose, autoimmune disease or viral infection. In one example, the subject may have been exposed to a toxin. The subject may have been diagnosed as suffering from an alcohol or acetaminophen overdose. It will be appreciated that the level of drug or alcohol in a subject which is defined as an overdose is known and can be calculated by the skilled medical practitioner.

Agents which are capable of inhibiting the interaction of podoplanin with CLEC-2, or inhibiting the activity of Src and/or Syk family kinases can be identified using functional assays known to the skilled person. Such assays may conveniently enable high throughput screening of potential inhibitor agents. For example, a protein-based assay can be derived by expressing and isolating proteins involved in the interaction of podoplanin with CLEC-2, and detecting the interaction of the proteins by ELISA. Potential inhibitor agents can be included in the ELISA. An inhibitory effect of an agent can then be detected by monitoring for reduced interaction between the proteins in the ELISA.

A transcription based assay can be derived by selecting transcriptional regulatory sequences (e.g. promoters) from genes involved in the CLEC-2-podoplanin pathway, and operatively linking such promoters to a reporter gene in an expression construct. The effect of different agents can then be detected by monitoring expression of the reporter gene in host cells transfected with the expression construct. One such assay is a luminescent reporter assay. Commonly used reporter genes include luciferase, beta-galactosidase, alkaline phosphatase and CAT (chloramphenicol acetyl transferase).

Other functional assays for detecting an inhibitory effect upon the interaction of podoplanin with CLEC-2 may include tyrosine kinase phosphorylation assays. Such assays will be known to the skilled person. For example, the skilled person may use src and/or syk phosphorylation assays. A reduction in Src and/or syk family kinase activation, measured by reduced phosphorylation downstream of Src or syk, may be used to detect the inhibitory effect of an agent upon the interaction of podoplanin with CLEC-2.

A platelet-aggregation assay can be derived by studying podoplanin-induced platelet aggregation in vitro in the presence of the agent. An inhibitory effect of the agent can then be detected from reduced platelet aggregation compared to control samples. The use of an ELISA and a platelet aggregation assay to monitor the inhibitory effect of an agent on the interaction of podoplanin with CLEC-2 is described by Nakazawa et al., Cancer Science, 2011 (102), 2051-2057.

Other functional assays for detecting an inhibitory effect may include measuring the affinity of the interaction between recombinant purified podoplanin and CLEC-2 in the presence or absence of the agent. The skilled person may use a Biacore X system and kit to measure the affinity, as described by Inoue et al., PLOS One, 2015, 10(9), 1-28. Thus, a reduction in affinity may be used to detect the inhibitory effect of the agent.

The agent may comprise or consist of a peptide, a protein, a truncated protein, an enzyme, an antibody or an antibody fragment (such as a Fab or F(ab′)₂ fragment, Fab-SH, an Fv antibody, an scFV antibody, a diabody or any other functional antigen-binding fragment), for example.

Agents which are peptides or proteins may be modified. For example, the peptide or protein may be PEGylated. Modified peptides or proteins may advantageously exhibit an improved circulatory half-life compared to non-modified peptides or proteins. The modification may be at the N and/or C terminus of the peptide or protein.

In some examples the agent may be a nucleic acid that specifically binds to CLEC-2 or podoplanin mRNA, thereby causing direct repression of expression of the gene to prevent translation into the CLEC-2 or podoplanin protein.

The agent may comprise or consist of a nucleic acid or a small molecule.

As used herein, a “small molecule” is a chemical compound having a molecular weight of no more than 900 daltons (Da). In some embodiments, the small molecule has a molecular weight of no more than 700 or no more than 500 Da. The small molecule may be an organic compound. The small molecule may bind to a protein component of the CLEC-2-podoplanin interaction and modulate its activity and/or interactions with other proteins or nucleic acids.

In some embodiments the agent comprises or consists of the small molecule 2CP, a derivative of 4-O-benzoyl- 3-methoxy-beta-nitrostyrene (BMNS). 2CP specifically binds to CLEC-2, as described by Chang et al., Oncotarget, 2015, 6(40), 42733-42748.

In some embodiments the agent comprises or consists of the small molecules fostamatinib, saracatinib or entospletinib.

In some embodiments the agent comprises or consists of use of kinase inhibitors. One such example of such is saracatinib a small molecule kinase inhibitor that inhibits the phosphorylation of key amino acids within kinases including syk.

In some embodiments the agent comprises or consists of an antisense molecule (e.g. an antisense DNA or RNA molecule or a chemical analogue) or a ribozyme molecule. Ribozymes and antisense molecules may be used to inhibit the transcription of a gene encoding CLEC-2 or podoplanin, or translation of the mRNA of that gene. Antisense molecules are oligonucleotides that bind in a sequence-specific manner to nucleic acids, such as DNA or RNA. When bound to mRNA that has a complementary sequence, antisense RNA prevents translation of the mRNA. Triplex molecules refer to single antisense DNA strands that bind duplex DNA forming a colinear triplex molecule, thereby preventing transcription. Particularly useful antisense nucleotides and triplex molecules are ones that are complementary to or bind the sense strand of DNA (or mRNA) that encodes a CLEC-2 or podoplanin protein.

In some embodiments, the agent comprises or consists of a short interfering nucleic acid (siNA). A siNA molecule may comprise a siDNA molecule or a siRNA molecule. In some embodiments, the agent comprises or consists of miRNA (microRNA), siRNA (small interfering RNA) or shRNA (short hairpin RNA). Oligonucleotides including siWAs can be prepared by solid phase chemical synthesis using standard techniques.

In embodiments wherein the agent is a peptide or protein, a nucleic acid sequence encoding the peptide or protein may be provided in a suitable vector, for example a plasmid, a cosmid or a viral vector. Thus, also provided is a vector (i.e. a construct), comprising a nucleic acid sequence which encodes the protein or peptide. The nucleic acid sequence is preferably operably linked to a suitable promoter. The invention further relates to a composition comprising the vector.

Agents which are nucleic acids, such as siRNAs or miRNAs, may be modified (e.g. via chemical modification of the nucleic acid backbone), or delivered in suitable delivery system which protects the nucleic acids from degradation and/or immune system recognition. Examples of suitable delivery systems include nanoparticles, lipid particles, polymer-mediated delivery systems, lipid-based nanovectors and exosomes.

In some embodiments the agent is a naturally occurring or a synthetic ligand of a protein involved in the interaction of podoplanin with CLEC-2, or Syk or Src kinase. The term “ligand” as used herein is understood to mean a substance that binds to a protein to form a complex. Formation of the complex may induce a change in the function or activity of the protein. A ligand may be an antagonist. As used herein, an “antagonist” is a molecule which binds to a protein and inhibits a biological response.

Proteins and peptides may be generated using a variety of methods, including purification of naturally-occurring proteins, recombinant protein production and de novo chemical synthesis.

In some embodiments the agent comprises or consists of a truncated protein. By “truncated” it will be appreciated that the protein lacks a portion of the full-length protein. The truncated protein may be inactive, or possess less activity as compared to the full length protein. As the skilled person will appreciate, the truncated protein may be capable of competitively binding to CLEC-2 or podoplanin.

In some embodiments the agent comprises or consists of truncated CLEC-2 or CLEC-1b. The truncated CLEC-2 or CLEC-1b may be capable of binding to podoplanin. The truncated CLEC-2 or CLEC-1b may lack at least a portion of an extracellular domain. In some embodiments the truncated CLEC-2 or CLEC-1b lacks a portion of the C-type lectin domain. The truncated CLEC-2 or CLEC-1b may lack at least a portion of the transmembrane domain and/or an N-terminal cytoplasmic tail. In some embodiments the truncated CLEC-2 or CLEC-1b lacks the transmembrane domain.

In some embodiments the agent comprises or consists of truncated podoplanin. The truncated podoplanin may be capable of binding to CLEC-2. The truncated podoplanin may lack at least a portion of the extracellular domain. The truncated podoplanin may lack at least a portion of the PLAG (platelet aggregation-stimulating) domain of podoplanin, for example at least one of PLAG1, PLAG2 or PLAG3. The truncated podoplanin may be derived from a splice variant, for example a naturally occurring splice variant.

In some embodiments the agent comprises an antibody or antibody fragment. In some embodiments the agent consists of an antibody or antibody fragment. The antibody may be monoclonal, polyclonal, recombinant or chimaeric. The term “chimaeric antibody” refers to an antibody consisting of antibody fragments derived from different species. Methods for generating antibodies are well-known to those skilled in the art. For example, the skilled person can use known hybridoma technology to generate and detect antibodies specific for CLEC2 or podoplanin. Commonly used assays to detect the specificity of an antibody for a particular target protein include ELISA, Western Blot and flow cytometry. Other methods to detect the specificity of an antibody will be known to the skilled person.

In some embodiments the agent comprises or consists of a humanised antibody. By “humanised” it will be appreciated that an antibody comprises or consists of human antibody fragments and antibody fragments from other species, for example rodents, e.g. mice. A humanised antibody may comprise human constant domains and variable domains from another species, for example rodent variable domains. In some embodiments a humanised antibody may comprise human variable and constant regions and rodent, for example mouse CDR (complementarity determining region) regions. Advantageously, humanised antibodies have reduced immunogenicity. In addition, humanised antibodies retain the high binding affinity of an antibody from a non-human species.

In some embodiments the agent is a human antibody or fragment thereof.

The agent may specifically bind to podoplanin. In some embodiments the agent comprises an antibody that specifically binds to podoplanin, i.e. an anti-podoplanin antibody or fragment. The generation and detection of an antibody specific for podoplanin is described by Nakazawa et al and Ogasawara et al, Monoclonal antibodies in Immunodiagnosis and Immunotherapy, 2016 (35), 1-8.

Antibodies that specifically bind to human podoplanin are provided in U.S. Pat. No. 8,697,073. Other suitable anti-podoplanin antibodies include LpMAb-13 (Ogasawara et al.), P2-0 or HAG-3 (Nakazawa et al). Commercially available anti-human podoplanin antibodies include the anti-human antibodies listed in Table 1. Commercially available anti-mouse podoplanin antibodies include the anti-mouse antibodies listed in Table 1. Known epitopes of human podoplanin are provided in U.S. Pat. No. 8,697,073. In some embodiments, the anti-podoplanin antibody specifically binds to at least one of the epitopes disclosed in U.S. Pat. No. 8,697,073, the epitope Ala42-Asp49 of human podoplanin, the PLAG1 epitope region of human podoplanin, the PLAG2 epitope region of human podoplanin the PLAG3 epitope region of human podoplanin or the PLAG4 epitope region of human podoplanin. In some embodiments, the anti-podoplanin antibody specifically binds to the 6 amino acid epitope sequence AMPGAE. In some embodiments, the anti-podoplanin antibody specifically binds to the 10 amino acid epitope sequence GVAMPGAEDD. Other suitable epitopes are provided by Ogasawara et al., Hybridoma, 2008, 27(4), 259-267

Known CDR regions of anti-podoplanin antibodies are also provided in U.S. Pat. No. 8,697,073.

In some embodiments the agent comprises or consists of an 8.1.1 clone hamster monoclonal anti-podoplanin antibody. This antibody is available commercially from various suppliers including, but not limited to Santa Cruz Biotechnology, AbCam, Biolegend, NovusBio and eBioscience. The antibody may specifically bind to mouse podoplanin. In one embodiment the agent comprises or consists of an NZ-1.3 clone rat monoclonal anti-podoplanin antibody. The antibody may specifically bind to human podoplanin. The NZ-1.3 clone rat monoclonal anti-podoplanin antibody is available commercially from at least eBioscience,

In some embodiments the agent specifically binds to CLEC-2. In some embodiments the agent comprises an antibody that binds specifically to CLEC-2, i.e. an anti-CLEC-2 antibody. The agent may comprise an antibody that binds specifically to human CLEC-2. The agent may comprise an antibody that binds specifically to rodent, for example mouse CLEC-2. Anti-human CLEC-2 antibodies are available from various suppliers including, but not limited to R&D Systems and Abcam.

As used herein, the terms “specifically binds to” or “specific for” will be understood to mean that the agent selectively recognises an epitope of a particular protein, for example, CLEC-2 or podoplanin.

Antibodies may be conjugated to other moieties, for example therapeutic or cytotaxic moieties. The conjugation of another moiety to an antibody advantageously allows the targeted delivery of an additional therapeutic moiety to CLEC-2, podoplanin, Src and/or Syk family kinases. This may serve to further inhibit the CLEC-2-podoplanin pathway. In other examples, antibodies may be conjugated to imaging moieties. The conjugation of an imaging moiety to an antibody advantageously allows the targeted imaging of the CLEC-2-podoplanin pathway, for example CLEC-2 or podoplanin. This may advantageously be used to visualise the in viva stage and/or the hepatic inflammation associated with acute liver failure.

Thus, in some embodiments, the agent comprises or consists of an antibody conjugate. The conjugate may comprise a cytokine or other molecule. In some embodiments the conjugate comprises a drug or radionuclide. Such antibody-conjugates are well-known in the art. In some embodiments the conjugate comprises a PET (position emission tomography) or MRI (magnetic resonance imaging) ligand. For example, the conjugate may comprise a PET ligand such a ⁶⁸ Gallium, ⁶⁴Cu or ²⁴I-labelled peptide or antibody. In other examples, the conjugate may comprise a MRI ligand such as a gadolinium contrast agent.

In some embodiments the agent is in combination with at least one additional agent. In some embodiments the at least one additional agent is selected from corticosteroids, N-acetyl cysteine (NAC), osmotic diuretics (e.g. mannitol), antidotes (e.g. penicillin G, silibinin, activated charcoal), barbiturate agents (e.g. pentobarbital, thiopental), benzodiazepines (e.g. midazolam), antibiotics, anaesthetic agents (e.g. propofol) or an agent that activates neutrophils.

Antibiotics may be broad spectrum and/or directed to gut infections, for example rifaximin.

In some embodiments the at least one additional agent is selected from corticosteroids, N-acetyl cysteine (NAC) or an agent that activates neutrophils.

In some embodiments the at least one additional agent is selected from corticosteroids or N-acetyl cysteine (NAC).

The agent and the additional agent may be administered concomitantly, sequentially or alternately.

Without wishing to be bound by theory, the present inventors propose that the use of an agent that inhibits the interaction of podoplanin with CLEC-2, or inhibits the activity of Src and/or Syk family kinases in combination with an additional agent has a synergistic effect in the treatment or prophylaxis of acute liver failure. Thus, the use of the agent in combination with at least one additional agent may further reduce liver failure and improve healing.

The agent may be administered at a timepoint of from 30 seconds to 200 hours post-diagnosis or post-onset of acute liver failure. In some embodiments, the agent is administered at a timepoint of at least 30 seconds, 1 minute, 5 minutes, 10 minutes, 30 minutes, 1 hour, 2 hours, 3 hours, 4 hours, 6 hours, 8 hours, 10 hours, 12 hours, 16 hours, 24 hours, 48 hours or 72 hours post-diagnosis or post-onset of acute liver failure. In some embodiments the agent is administered at a timepoint of no more than 200 hours, 150 hours, 120 hours, 100 hours, 72 hours, 48 hours or 24 hours post-diagnosis or post-onset of acute liver failure. The agent may be administered at a timepoint of from 30 seconds to 48 hours post-diagnosis or post-onset of acute liver failure. In some embodiments, the agent is administered at a timepoint of from 1 to 72 hours post-diagnosis or post onset of acute liver failure. In some embodiments the agent, is administered at a timepoint of from 6 to 72 hours post-diagnosis or post-onset of acute liver failure. In some embodiments the agent is administered at a timepoint of from 24 to 48 hours post-diagnosis or post-onset of acute liver failure, or of from 48 to 72 hours post-diagnosis or post-onset of acute liver failure. In some embodiments the agent is administered at a timepoint of from 30 minutes to 72 hours post diagnosis or post-onset, or at from 30 minutes to 24 hours post diagnosis or post-onset.

In some embodiments the agent is administered at from 30 seconds to 72 hours, such as between 20 -30 hours (such as 24 or 28 hours) post-diagnosis or post-onset of acute liver failure.

In some embodiments, the agent is administered prior to diagnosis of acute liver failure.

In some embodiments the agent is administered at a dose of between 0.1 μg/kg of body weight and 1 g/kg of body weight, depending upon the specific agent used. In some embodiments the agent is administered at a dose of at least 0.1 μg/kg of body weight, 0.2 μg/kg of body weight, 0.3 μg/kg of body weight, 0.5 μg/kg of body weight, 1 μg/kg of body weight, 5 μg/kg of body weight, 10 μg/kg of body weight, 50 μg/kg of body weight, 100 μg/kg of body weight, 150 μg/kg of body weight, 200 μg/kg of body weight, 500 μg/kg of body weight, 1000 μg/kg of body weight, 2000 μg/kg of body weight or 5000 μg/kg of body weight. In some embodiments the agent is administered at a dose of no more than 50000 μg/kg of body weight, 25000 μg/kg of body weight, 10000 μg/kg of body weight, 7000 μg/kg of body weight, 5000 μg/kg of body weight, 2000 μg/kg of body weight, 1000 μg/kg of body weight, 500 μg/kg of body weight, 200 μg/kg of body weight, 150 μg/kg of body weight, 100 μg/kg of body weight, 50 μg/kg of body weight or 10 μg/kg of body weight. In some embodiments the agent is administered at a dose of between 10000 μg/kg of body weight and 0.5 g/kg of body weight, depending upon the specific agent used. In some embodiments the agent is administered at a dose of between 10000 μg/kg of body weight and 100000 μg/kg of body weight, depending upon the specific agent used, hi some embodiments the agent is administered at a dose of between 0.1 g/kg of body weight and 0.5 g/kg of body weight, depending upon the specific agent used.

As the skilled person will appreciate, acute liver failure may require rapid or immediate treatment. Failure to do so could result in increased liver failure, reduced healing and/or increased morbidity or mortality. It is therefore important to administer the agent either prior to or soon after diagnosis. The rapid administration of the agent also gives the skilled medical practitioner sufficient time to assess the efficacy of the agent in the treatment of acute liver failure in order to determine if further therapeutic intervention, for example, transplantation, is required.

According to a second aspect of the invention there is provided the use of an agent that inhibits the interaction of podoplanin with CLEC-2 or inhibits the activity of Src and/or Syk family kinases in the manufacture of a medicament for the treatment and/or prophylaxis of acute liver failure in a subject.

According to a third aspect of the invention there is provided a composition comprising a therapeutically effective amount of an agent that inhibits, the interaction of podoplanin with CLEC-2 or inhibits the activity of Src and/or Syk family kinases, wherein said therapeutically effective amount is sufficient to eliminate, reduce or prevent acute liver failure.

As used herein, a “therapeutically effective amount” is an amount of the agent that inhibits the interaction of podoplanin with CLEC-2 or inhibits the activity of Src and/or Syk family kinases which, when administered to a subject, is sufficient to eliminate, reduce or prevent acute liver failure. A therapeutically effective amount may also be an amount at which there are no toxic or detrimental effects, or a level at which any toxic or detrimental effects are outweighed by the therapeutic benefits.

The composition may further comprise a pharmaceutically acceptable carrier, diluent or excipient. A “pharmaceutically acceptable carrier” as referred to herein is any physiological vehicle known to those of ordinary skill in the art useful in formulating pharmaceutical compositions. A “diluent” as referred to herein is any substance known to those of ordinary skill in the art useful in diluting agents for use in pharmaceutical compositions. The agent may be mixed with, or dissolved, suspended or dispersed in the carrier, diluent or excipient.

The composition may be in the form of a capsule, tablet, liquid, ointment, cream, gel, hydrogel, aerosol, spray, micelle, transdermal patch, liposome or any other suitable form that may be administered to a mammal suffering from, or at risk of developing acute liver failure.

The composition may comprise the agent at a concentration of up to 100 μm.

Administration of the agent may be by any suitable route, including but not limited to, injection (including intravenous (bolus or infusion), intra-arterial, intraperitoneal, subcutaneous (bolus or infusion), intraventricular, intramuscular, or subarachnoidal), oral ingestion, inhalation, topical, via a mucosa (such as the oral, nasal or rectal mucosa), by delivery in the form of a spray, tablet, transdermal patch, subcutaneous implant or in the form of a suppository.

The agent may be administered as a single dose or as multiple doses. Multiple doses may be administered in a single day (e.g. 2, 3 or 4 doses at intervals of e.g. 3, 6 or 8 hours). The agent may be administered on a regular basis (e.g. daily, every other day, or weekly) over a period of days, weeks or months, as appropriate.

It will be appreciated that optimal doses to be administered can be determined by those skilled in the art, and will vary depending on the particular agent in use, the strength of the preparation, the mode of administration, the advancement or severity of the acute liver failure, and the cause of the acute liver failure. Additional factors depending on the particular subject being treated will result in a need to adjust dosages, including subject age, weight, gender, diet, and time of administration. Known procedures, such as those conventionally employed by the pharmaceutical industry (e.g. in vivo experimentation, clinical trials, etc.), may be used to establish specific formulations for use according to the invention and precise therapeutic dosage regimes.

In some embodiments, the composition comprises at least one additional agent. The additional agent may be selected from corticosteroids, N-acetyl cysteine (NAC), osmotic diuretics (e.g. mannitol), antidotes (e.g. penicillin G, silibinin, activated charcoal), barbiturate agents (e.g. pentobarbital, thiopental), benzodiazepines (e.g. midazolam), anaesthetic agents (e.g. propofol) or an agent that activates neutrophils.

In some embodiments the at least one additional agent is selected from corticosteroids, N-acetyl cysteine (NAC) or an agent that activates neutrophils.

In some embodiments the at least one additional agent is selected from corticosteroids or N-acetyl cysteine (NAC).

According to a further aspect of the invention there is provided a composition comprising a therapeutically effective amount of a combination of an agent that inhibits the interaction of podoplanin with CLEC-2 or inhibits the activity of Src and/or Syk family kinases and at least one additional agent, wherein said therapeutically effective amount is sufficient to eliminate, reduce or prevent acute liver failure.

In some embodiments the at least one additional agent is selected from corticosteroids, N-acetyl cysteine (NAC) or an agent that activates neutrophils.

In some embodiments the at least one additional agent is selected from corticosteroids or N-acetyl cysteine (NAC).

According to a fifth aspect of the invention there is provided a method for the treatment or prophylaxis of acute liver failure in a subject, the method comprising the administration of an agent that inhibits the interaction of podoplanin with CLEC-2 or inhibits the activity of Src and/or Syk family kinases to said subject.

The method may comprise the administration of a therapeutically effective amount of the agent. The method may comprise administering the agent at from 30 minutes to 200 hours post-diagnosis or post-onset of acute liver failure. The method may comprise administering the agent at from 1 to 72 hours post-diagnosis or post-onset of acute liver failure. In some embodiments the method comprises administering the agent at from 6 to 72 hours post-diagnosis or post-onset of acute liver failure. In some embodiments the method comprises administering the agent at from 24 to 48 hours post-diagnosis or post-onset of acute liver failure, or at from 48 to 72 hours post-diagnosis or post-onset of acute liver failure. In some embodiments the method comprises administering the agent at from 30 minutes to 72 hours post diagnosis or post-onset, or at from 30 minutes to 24 hours post diagnosis or post-onset.

In some embodiments the method comprises administering the agent at from 24 to 72 hours post-diagnosis or post-onset of acute liver failure.

The method may further comprise liver dialysis and/or administration of agents directed against the podoplanin pathway as discussed herein, for example using a molecular adsorbents recirculation system (MARS), Single Pass Albumin Dialysis (SPAD), continuous veno-venous haemodiafiltration (CVVHDF) or a Prometheus system.

In some embodiments the method comprises the administration of the agent prior to diagnosis of acute liver failure.

The method may comprise administering a dose of the agent of between 0.1 μg/kg of body weight and 1 g/kg of body weight of the agent. In some embodiments the method comprises administering a dose of the agent of at least 0.1 μg/kg of body weight, 0.2 μg/kg of body weight, 0.3 μg/kg of body weight, 0.5 μg/kg of body weight, 1 μg/kg of body weight, 5 μg/kg of body weight, 10 μg/kg of body weight, 50 μg/kg of body weight, 100 μg/kg of body weight, 150 μg/kg of body weight, 200 μg/kg of body weight, 500 μg/kg of body weight, 1000 μg/kg of body weight, 2000 μg/kg of body weight or 5000 μg/kg of body weight. In some embodiments the method comprises administering a dose of the agent of no more than 50000 μg/kg of body weight, 25000 μg/kg of body weight, 10000 μg/kg of body weight, 7000 μg/kg of body weight, 5000 μg/kg of body weight, 2000 μg/kg of body weight, 1000 μg/kg of body weight, 500 μg/kg of body weight, 200 μg/kg of body weight, 150 μg/kg of body weight, 100 μg/kg of body weight, 50 μg/kg of body weight or 10 μg/kg of body weight. In some embodiments the method comprises administering a dose of the agent of between 10000 μg/kg of body weight and 0.5 g/kg of body weight. In some embodiments the method comprises administering a dose of the agent of between 10000 μg/kg of body weight and 100000 μg/kg of body weight. The method may comprise administering a dose of the agent of between 0.1 g/kg of body weight and 0.5 g/kg of body weight.

The method may comprise administering the agent, as a single dose or as multiple doses, Multiple doses may be administered in a single day (e.g. 2, 3 or 4 doses at intervals of e.g. 3, 6 or 8 hours). The agent may be administered on a regular basis (e.g. daily, every other day, or weekly) over a period of days, weeks or months, as appropriate.

One known method for the treatment of acute liver failure is liver transplantation. Thus, the agent may be administered before, during or after liver transplantation to said subject. In the context of liver transplantation, “before” will be understood to refer to prior to the start of surgery. “During” will be understood to refer to administration between the start and end of surgery. “After” will be understood to refer to the administration after the end of surgery. The agent may be administered no more than 30 minutes, no more than 1 hour, no more than 2 hours, no more than 3 hours, no more than 4 hours, no more than 6 hours, no more than 12 hours, no more than 24 hours, no more than 48 hours or no more than 72 hours before liver transplantation. The agent may be administered no more than 30 minutes, no more than 1 hour, no more than 2 hours, no more than 3 hours, no more than 4 hours, no more than 6 hours, no more than 12 hours, no more than 24 hours, no more than 48 hours or no more than 72 hours after liver transplantation In some embodiments the agent is administered to the transplanted liver, or the site into which the transplanted liver will be placed. It will be appreciated that the administration of the agent before, during or after liver transplantation may reduce transplant rejection. Administration of the agent before, during or after liver transplantation may also improve the subject's recovery time and improve transplant integration and/or healing in the subject.

According to a sixth aspect of the invention there is provided a method of determining the efficacy of treatment of acute liver failure in a subject using an agent that inhibits the interaction of podoplanin with CLEC-2 or inhibits the activity of Src and/or Syk family kinases, the method comprising isolating samples from the subject; and determining in the samples whether the levels of alanine transaminase (ALT) have decreased after the treatment.

The method for determining the efficacy may comprise determining whether the levels of one or more additional characteristic serological or clinical parameters of liver health have normalised in blood after the treatment. Normalisation will be understood to refer to a modification of levels of one or more characteristic serological parameters to normal levels.

The one or more characteristic serological parameters may include INR, aminotransferases, bilirubin, serum lactate, serum pH, renal function (creatinine/urea), sodium, ammonia, CRP (C-reactive protein), ESR (erythrocyte sedimentation rate) and/or albumin.

Normal levels of the one or more characteristic serological parameters will be known to the skilled medical practitioner. For the avoidance of doubt, it will be understood that “normalisation” of INR (International Normalised Ratio) will be considered to be a decrease towards baseline. Normalisation of aminotransferases will be considered to be a decrease from levels of over 500 IU/L towards normal levels. Normalisation of bilirubin will be considered to be a decrease towards normal levels, and normalisation of albumin will be considered to be an increase towards normal levels. Normalisation of serum lactate or serum pH will be considered to be an increase in levels.

The one or more characteristic clinical parameters may include resolution or improvement of hepatic encephalopathy, improvement in end organ perfusion measured by an improvement in GCS (Glasgow Coma Scale), improvement in urine output, maintenance of adequate mean arterial blood pressure (MAP), adequate organ oxygenation (measured by partial pressure of oxygen in arterial blood), improvement in intracranial pressures, reduction in portal pressures, resolution or reduction in size of ascites, resolution of sepsis (improvement in markers for systemic inflammatory response syndrome including temperature, pulse, blood pressure and respiratory rate) and/or reduced dependence on life support systems/drugs such as invasive or non-invasive ventilation, ionotropic blood pressure support, blood filtering systems including renal dialysis and MARS and/or nutritional support.

The method may also comprise the assessment of composite scores such as the Kings criteria, Clichy criteria or intensive care scores, for example SOFA (Sepsis-related Organ Failure Assessment Score) or APACHE (Acute Physiology and Chronic Health Evaluation) scores. Efficacy of treatment may be considered to be an improvement in composite score or scores.

Tests for the diagnosis of acute liver failure may include coagulation studies, the detection of aspartate aminotransferase (AST)/serum glutamic-oxaloacetic transaminase (SGOT), alanine aminotransferase (ALT)/serum glutamic-pyruvic transaminase (SGPT), alkaline phosphatase (ALP), glucose, bilirubin, ammonia, lactate, phosphate, creatinine, or copper and/ceruloplasmin levels in a sample from the subject. The skilled practitioner may assess the levels and/or acetaminophen-product adduct levels in a sample from a subject. Other diagnosis methods may include viral serologies, the detection of autoimmune markers, electroencephalography, intracranial pressure monitoring, liver biopsy or imaging. Viral serologies may include the detection of viral surface antigen, or Immunoglobulin, for example, the detection of hepatitis A, B, C, D or E virus immunoglobulin M (IgM) or hepatitis B surface antigen (HbsAg). Liver biopsy may be percutaneous or transjugular. Imaging may include hepatic doppler ultrasonography, abdominal computed tomography (CT) scanning, magnetic resonance imaging or cranial CT scanning. Levels to be detected may be increased or decreased relative to normal levels.

All of the features described herein (including any accompanying claims, abstract and drawings) may be combined with any of the above aspects in any combination, unless otherwise indicated.

### DETAILED DESCRIPTION OF THE INVENTION

Embodiments of the invention will now be described by way of example and with reference to the accompanying Figures:

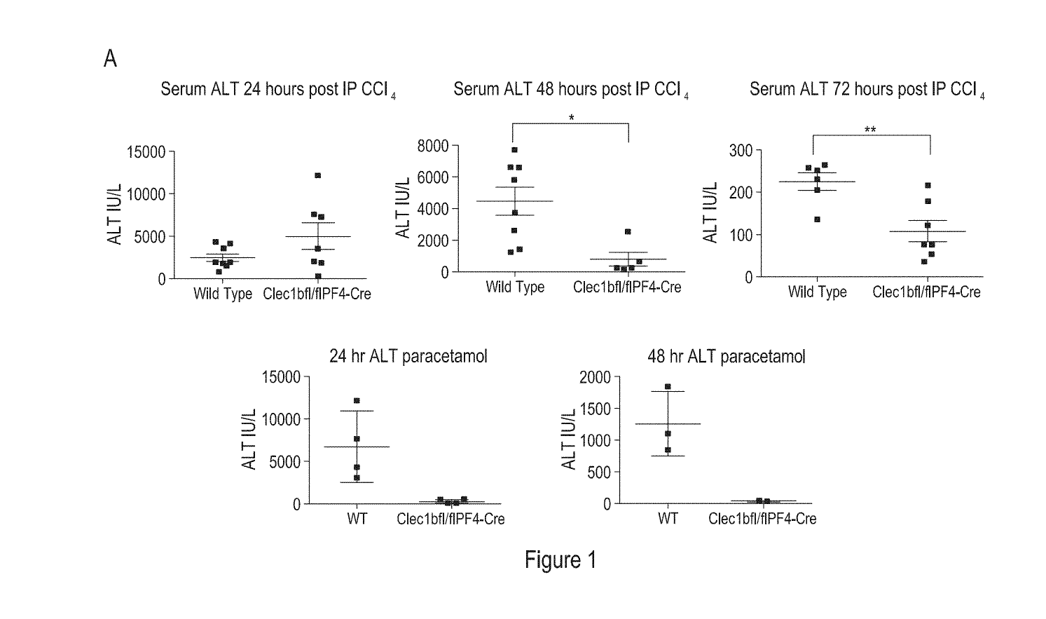

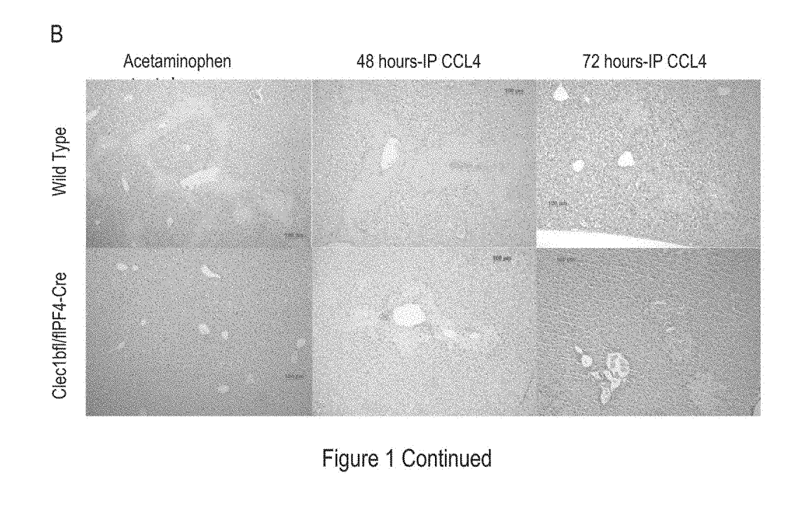

FIG. 1 shows: Mice with CLEC-2 deficient platelets (CLEC1 b fl/fl PF4cre) exhibit highly enhanced healing after a toxic liver injury. Wild-type or CLEC-2 deficient mice were injected intraperitoneally with carbon tetrachloride or acetaminophen (paracetamol) and sacrificed ether 24, 48 or 72 hours after injection. (A) Serum alanine transaminase levels (ALT) at 24, 48 or 72 hours post injection of wild type (WT) and CLEC1b fl/fl PF4 cre mice (n=5-8 per group) (*P<0.05, ***P <0.01, ***P <0.001). (B) Hematoxylin-eosin staining of liver tissue sections.

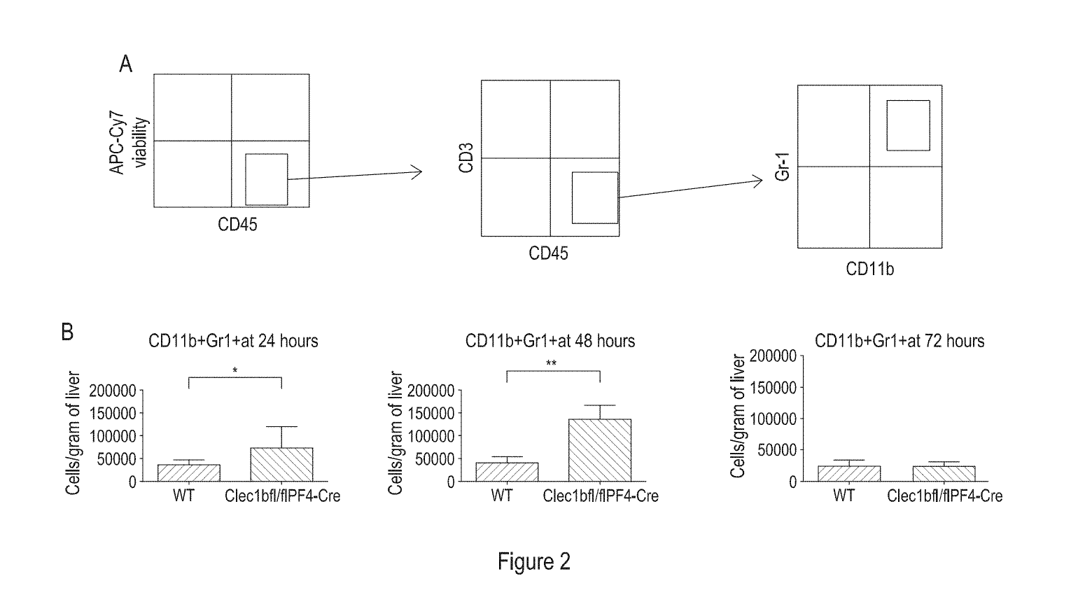

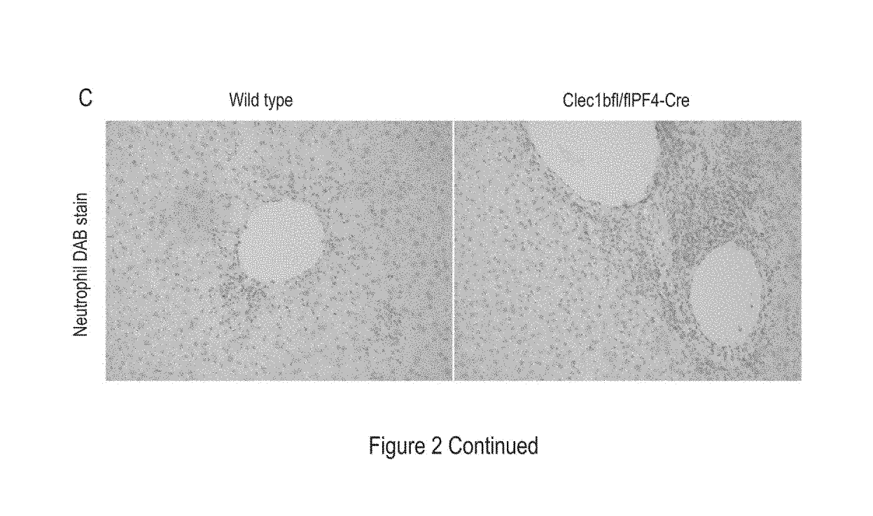

FIG. 2 shows: CLEC1b fl/fl PF4cre mice exhibit greater hepatic neutrophil recruitment than wild type animals after CCL4 injection. Livers from carbon tetrachloride or paracetamol-injured mice were sacrificed at 24, 48 or 72 hours post injection. Isolated livers were digested, CD11b⁺Gr1⁺cells (neutrophils) were isolated and the number of cells per gram of liver tissue (n=5-8 per group) was quantified by flow cytometry. (A) Gating strategy to define CD11b⁺GR1⁺ cells. (B) Number of neutrophils per gram of liver tissue in WT and CLEC1b fl/fl PF4cre mice (*P<0.05, **P<0.01, ***P<0.001). (C) Liver sections obtained from mice 72 hours post injection were stained with antibody against neutrophil elastase and visualised using a DAB stain (positive staining indicated in brown, sections counterstained using haematoxylin), representative portal fields from WT and CLEC1b fl/fl PF4cre are shown at 20× magnification.

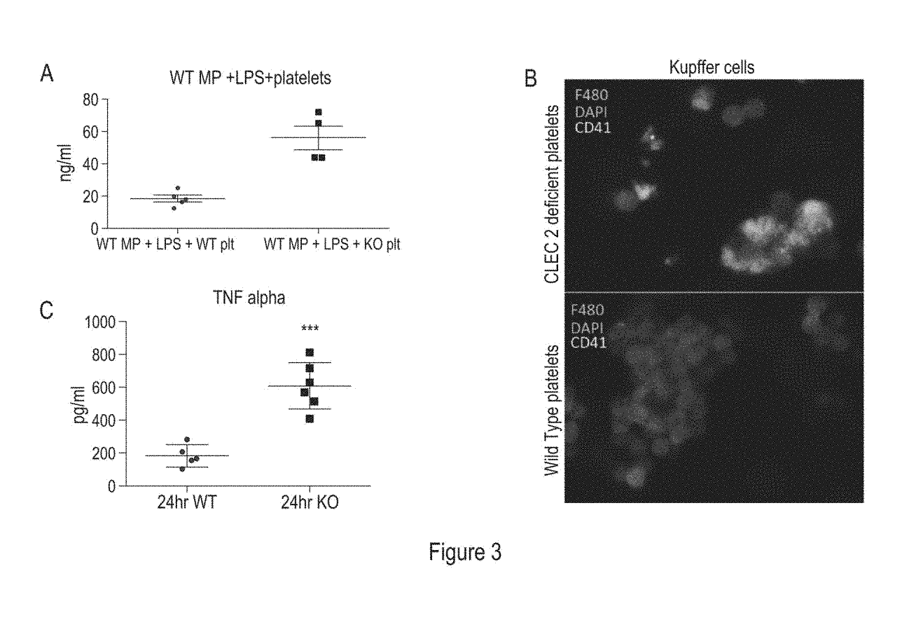

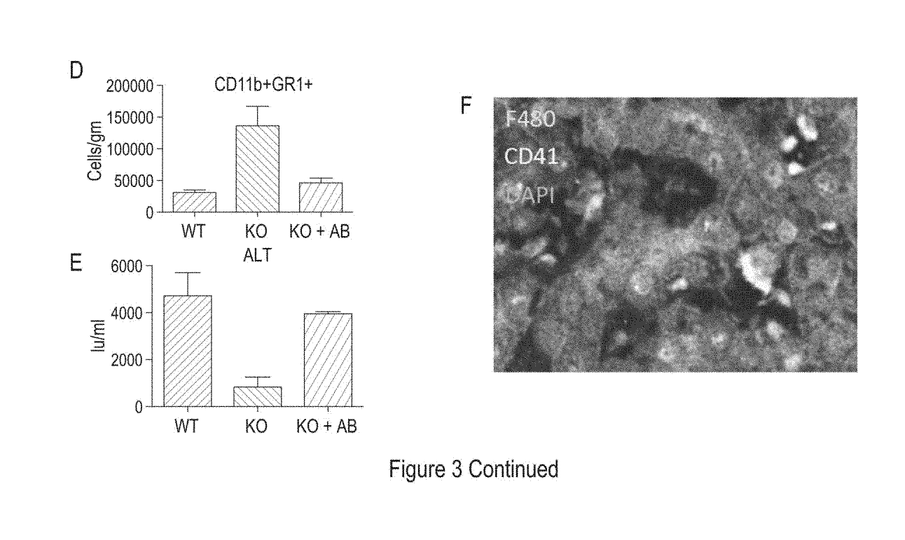

FIG. 3 shows: CLEC-2 deficient platelets interact with Kupffer cells and enhance TNF-alpha production, thus increasing neutrophil recruitment in CLEC1b fl/fl PF4cre (KO) mice. Kupffer cells isolated from WT mouse livers were plated in a tissue culture well, treated with lipopolysaccharide (LPS) and incubated with either platelets from CLEC2 deficient animals or WT platelets. (A) Production of TNF-alpha by Kupffer cells was measured in response to LPS plus either CLEC-2 deficient (KO) platelets or WT platelets (n=4 per group). (B) Isolated Kupffer cells (F480⁺, shown in green), were incubated with either CLEC2 deficient or WT platelets (CD41⁺, shown in purple). DAPI (blue) was used as a nuclear stain. Representative images (63× magnification) are shown. (C) Serum was isolated from WT and CLEC1b fl/fl PF4cre (KO) mice at 24 hours after carbon tetrachloride injection and serum TNF-alpha levels measured by ELISA. Levels are shown as picograms/ml (n=6/group) (*P<0.05, **P<0.01, ***P<0.001), (D) CLEC1b fl/fl PF4cre mice were pre-treated with an anti-TNF-alpha monoclonal antibody (Etanercept) before carbon tetrachloride injection (KO+AB). WT and CLEC1b fl/fl PF4cre mice not pre-treated before carbon tetrachloride injection were used as controls (WT and KO, accordingly). Mice were sacrificed 48 hours after the carbon tetrachloride injection. Data shown represents neutrophils per gram of liver tissue in either WT, CLEC1b fl/fl PF4cre (KO) or Etanercept treated CLEC1b fl/fl PF4cre mice (KO+AB) (n=2-6 per group). (E) Serum ALT from the groups in (D) at the same time point (48 hours post carbon tetrachloride) is shown. (F) Frozen mouse liver tissue from CLEC1b PF4cre (KO) mice was stained for Kupffer cells (F480+, shown in purple), and platelets (CD41⁺, shown in yellow). DAPI was used as a nuclear counterstain (blue).

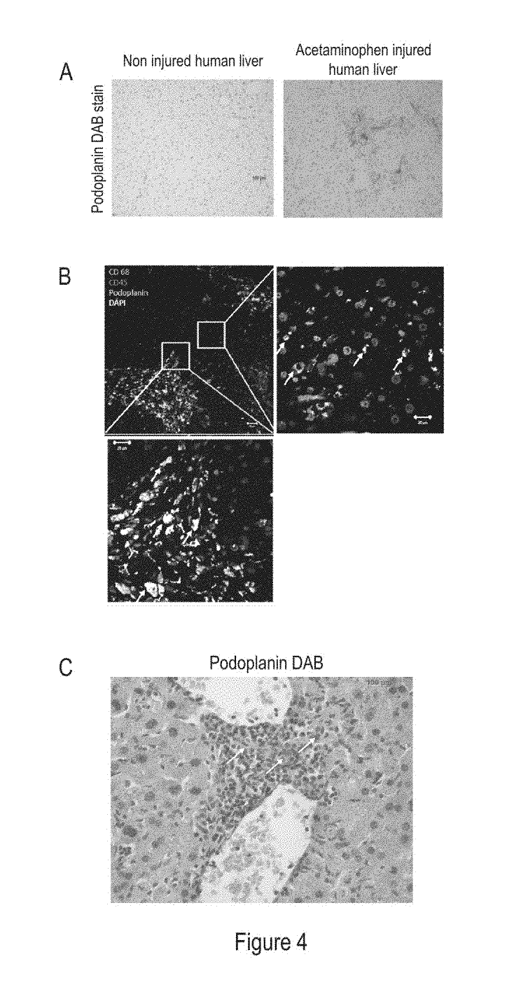

FIG. 4 shows: Podoplanin is upregulated during toxic injury by macrophages in human and mouse livers. (A) Podoplanin (shown in brown) is upregulated on cells within the inflammatory filtrate during acetaminophen (paracetamol) induced human liver injury but not in uninjured control liver. (B) The cells which express Podoplanin in human acetaminophen-induced liver injury are hepatic macrophages or Kupffer cells. These cells (white arrows) are shown as sea green as they co-express podoplanin (blue) and CD68 (marker of monocytes and tissue macrophages, shown in green). (C) Livers were isolated 48 hours post CCL4 injection. The cellular infiltrate within these injured mouse livers expresses podoplanin (pink).

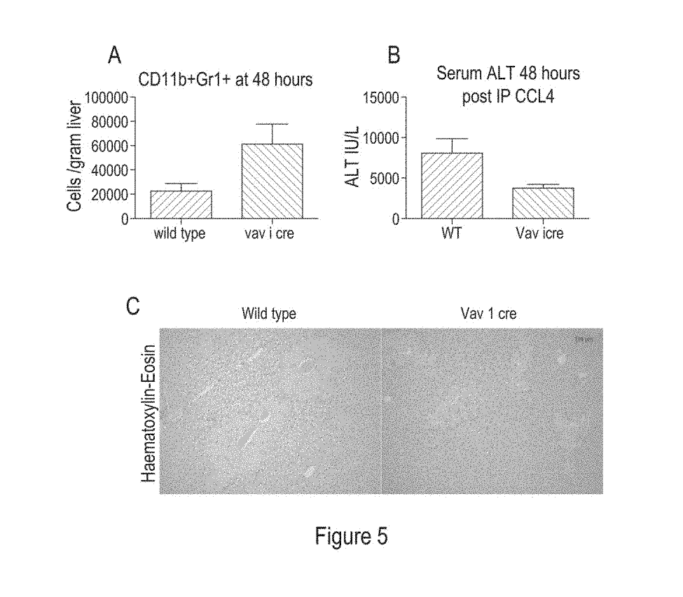

FIG. 5 shows: Podoplanin deficient mice exhibit enhanced neutrophil recruitment and reduced liver failure compared to wild-type mice. WT and Vav-1 cre (podoplanin deficient mice) were injected with carbon tetrachloride and sacrificed 48 hours after injection. (A) Isolated livers were digested, CD11b⁺Gr1⁺cells (neutrophils) were isolated and the number of cells per gram of liver tissue (n=4 per group) was quantified by flow cytometry. (B) ALT levels in serum isolated 48 hours after injection with carbon tetrachloride in WT and Vav-1 cre mice are shown. (C) Representative haematoxylin and eosin staining of liver tissue from mice (WT and podoplanin-deficient) injured with carbon tetrachloride and collected 48 hours later. Areas of tissue necrosis are indicated by pink eosin staining, and are reduced in the Vav1 cre (podoplanin deficient) liver.

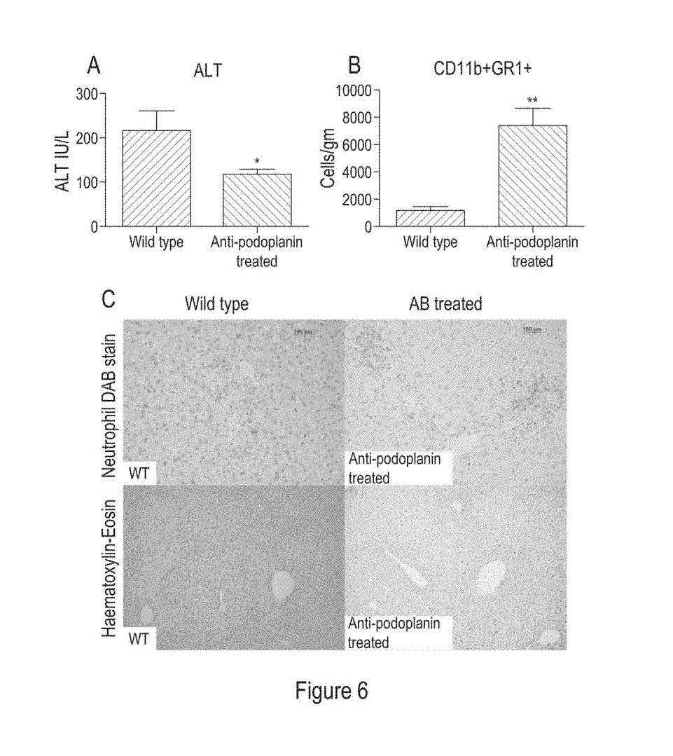

FIG. 6 shows: A selective podoplanin function-blocking antibody reduced liver injury by enhancing neutrophil recruitment after carbon tetrachloride induced liver injury. Mice were treated with an intravenous podoplanin blocking antibody (anti-podoplanin) prior to carbon tetrachloride injection. Mice were sacrificed 72 hours after carbon tetrachloride injection. (A) Serum ALT levels at time of sacrifice are shown, WT or antibody-treated groups were compared (n=6 per group) (*P<0.05, **P<0.01, ***P<0.001). (B) Number of neutrophils per gram of liver tissue from WT and antibody-treated mice (*P<0.05. **P<0.01, ***P<0.001). (C) Liver tissue from WT or anti-podoplanin treated mice was stained with a neutrophil elastase DAB stain or Haematoxylin-Eosin.

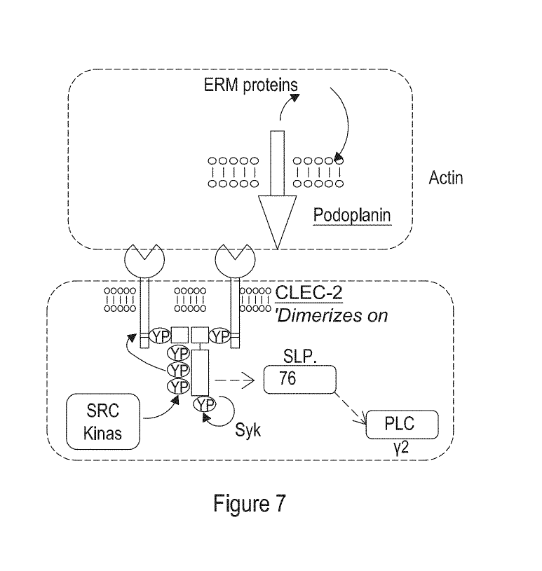

FIG. 7 shows: The CLEC2-Podoplanin interaction, Podoplanin and Clec-2 are expressed on the membrane of key cell populations such as macrophages and platelets respectively. Podoplanin has a single transmembrane region and short cytoplasmic tail that interacts with members of the ERM family of proteins to link podoplanin to the actin cytoskeleton. Binding of podoplanin, the only known physiological ligand for CLEC-2 results in phosphorylation of tyrosine residues in a YXXL motif in the intracellular ITAM domain of CLEC-2 and permits CLEC-2 to interact with tyrosine kinases such as SRC and Syk. This leads to activation of other downstream partners such as SLP-76 and PLCy and causes platelet activation and aggregation. Of note the interaction with Syk is mediated by a single YXXL motif (or HemilTAM) within the cytoplasmic tail of CLEC-2 and thus dimerization of CLEC-2 in response to ligand binding facilitates the signal transduction activity via Syk. In addition Tyrosine phosphorylation of the hemilTAM domain is mediated by an interplay between Src and Syk tyrosine kinases.

Table I shows: Commercially available anti-podoplanin antibodies.

### Examples

Platelets are fundamental players in liver pathobiology; driving inflammation, fibrosis, cancer and even aiding regeneration. However, the specific molecular basis of platelet activation in the context of liver inflammation and failure remains elusive.

The present inventors thus sought to explore the molecular basis of platelet activation in liver inflammation and failure.

### Materials and Methods

### Mice

C57BL/6J mice were obtained from Harlan OLAC LTD or from in-house colonies. VaviCre⁺-Podoplanin^{fl/fl} mice (obtained from Jackson Laboratories) and PF4Cre-CLEC-2^{fl/fl} mice are described in Finney et al., Blood, 2012 (119), 1747-1756. All strains of genetically-altered mice are on a C57BL/6J background. Control mice were matched by genetic background, age and sex. All mice were housed at the Biomedical Services Unit, University of Birmingham and used under procedure in accordance with UK Home Office guidelines.

### Human Tissue

Human liver was collected from patients in the liver transplantation programme at Queen Elizabeth Hospital in Birmingham. All samples were collected with written informed patient consent and under local ethical approvals. Normal liver tissue was obtained from donor tissue that was surplus to requirement for transplantation, or deemed unsuitable for use. Diseased liver tissue was from explanted livers collected during transplantation surgery.

### Induction of Liver Injury

Acute hepatic inflammation was induced using intraperitoneal injections of CCl₄(carbon tetrachloride) (Sigma-Aldrich) or acetaminophen (Sigma-Aldrich). CCl₄ was diluted 1:4 with mineral oil, and injected intraperitoneally into mice at a concentration of 1 ml/kg (control animals were treated with IP mineral oil alone. Acetaminophen was dissolved in phosphate buffered saline (PBS) (Dulbecco) at a temperature of 60° C. The solution was cooled to 37° C. prior to injection and the final concentration injected was 350 mg/Kg. Control mice received intraperitoneal injections of PBS only.

### Antibody Treatment

Mice were pre-treated with 100 μg of functional grade purified anti-podoplanin 8.1.1 intravenously 24 hours prior to being intraperitoneally injected with either acetaminophen or CCl₄.

### Immunohistology and Confocal Microscopy

Tissues were snap-frozen or fixed in 4% Formaldehyde immediately upon removal. Paraffin-embedded tissue sections were stained by Haematoxylin and Eosin.

Mouse frozen tissue sections were stained by immunohistochemistry (IHC) to detect podoplanin (eBio8.1.1, eBioscience), F4/80, neutrophil elastase and platelets, using methodology as described previously in Bowman et al., Am. J Pathol., 2014 (184), 150-1561.

Human frozen tissue sections were stained by immunohistochemistry to detect podoplanin and CD68. IHC was performed in Tris buffer (pH 7.6). Primary and secondary antibodies were added for 60 and 45 minutes respectively at room temperature. Horse-radish peroxidase conjugated secondary antibodies were developed using Alkaline-phosphatase (ABComplex, Vector Laboratories) and 3,3′-diaminobenzidine tetrahydrochloride. Slides were mounted in DPX and images acquired at ×20 or ×10 magnification using a Leica CTR6000 microscope (Leica, Milton-Keynes, UK), with Qcapture software. Low magnification images were acquired by a Carl Zeiss AxioScan.Z1 Slide Scanner using a 3CCD colour 2MP Hitachi 1200×1600 HV-F202SCL camera. Images were analysed using Zen blue (2012) slide scan software.

Fluorescent confocal microscopy was performed on frozen liver sections: CD41 (MWReg30), CD68, CD31 and podoplanin (eBio8.1.1) using methods as previously described (Weston et al., J Clin Invest, 2015(125), 501-520. Staining was performed in PBS+1% FCS. Sections were incubated with primary and secondary antibodies for 9 and 4 minutes respectively at room temperature in the dark. Nuclei were detected by Hoechst 33342 (10 μg/ml for 2 minutes at room temperature). Slides were mounted using Prolong Gold Anti-fade reagent (Invitrogen, Paisley, UK), and images were taken using either a ×10, ×40 or ×63 magnification objective on a LSM510 laser scanning confocal microscope with a Zeiss AxioVert 100M (Zeiss, Germany) in conjunction with Zeiss LSM image software.

### Quantification of Liver-Infiltrating Immune Cells

Mouse livers were harvested after the animal was euthanized under deep sedation after cardiac puncture. The organs were then weighed and dissociated in a gentleMACS C Tube (Miltenyi Biotec). The resulting immune cells were then purified using an Optiprep gradient (Sigma) and analysed by flow cytometry. Inflammatory cells were gated as a CD4+ cell population (anti-0045-PerCP-Cy5., clone 30-F11; BD Biosciences), and non-viable cells were excluded using a Zombie NIR™ Fixable Viability kit (BioLegend). Lymphocytes were characterised based on staining using a cocktail of anti-CD3 Pacific blue (clone 500A2); anti-CD4-PE (clone RM4-5): anti-CD8a-APC (clone 53-6.7): anti-CD19-APC-Cy7 or anti-CD19-BV510 (both clone 1D3); and anti-NK1.1-FITC (clone PK136) or DX5-FITC (clone DX5) abs (all from BD Biosciences). The monocyte subsets were identified by staining with anti-CD11b-PE (clone M1170; BD Biosciences); anti-GR1-APC (clone RBS-805; BD Biosciences); and anti-F4/80-FITC (clone BM8; eBioscience) abs. Absolute cell counts were determined with AccuCheck Counting Beads (Invitrogen), and the number of cells was normalised to the total liver weight. Data were analysed using a CyAn ADP flow cytometer (Beckman Coulter) or a BD LSRII using Summit version 4.3 or FlowJo version 10.0.7 software where appropriate.

### Kupffer Cell Isolation

Kupffer cells were isolated from murine livers using Blomhoff's method of selective plastic adherence. As described above, cell suspensions obtained from murine livers were subjected to gradient centrifugation. In detail, cell sediments were re-suspended with 10 ml RPMI 1640 and centrifuged at 300×g for 5 min at 4° C., the top aqueous phase was discarded, and the cell sediments were reserved. The cell sediments were then re-suspended with 10 ml RPMI 1640 and centrifuged at 50×g for 3 min at 4° C. The top aqueous phase (cleared cell suspension) was transferred into a new 10 ml centrifuge tube and centrifuged at 300×g for 5 min at 4° C. The top aqueous phase was discarded and the cell sediments were reserved. The cell sediments mainly contained non-parenchymal cells of the liver that were KCs, sinusoidal endothelial cells and satellite cells. To purify the obtained cell population further, the method of selective adherence to plastic was used according to Blomhoff et al., Methods in Enzymology, Vol. 190, 58-71. The cells were then seeded into six-well plates at a density of 1-3×10⁷/well in Dulbecco's Modified Eagle's Medium (DMEM, Hyclone, USA), supplemented with 10% feta bovine serum (FBS, Hyclone, USA), and 100 U/ml Penicillin/Streptomycin (Sigma, USA), and incubated for 2 hrs in a 5% CO₂ atmosphere at 37° C. Non-adherent cells were then removed from the dish by gently washing with PBS, the adherent cells were Kupffer cells.

### Biochemical Liver Injury Assays

Serum was isolated from whole blood and levels of liver-specific enzymes AST and ALT were measured on a clinical autoanalyser, according to standard protocols in the Biochemistry Department at the Birmingham Women's Hospital, Birmingham, UK.

### INF-Alpha ELISA

TNF-alpha levels were determined from either mouse serum or macrophage cell culture supernatant by ELISA according to the manufacturer's instructions (eBioscience: Ready Set Go—TNF ELISA). TNF concentration in serum/supernatant was calculated compared to a calibration curve and expressed as μg/ml.

### Results

### Enhanced Healing after a Toxic Liver Injury in Mice with CLEC-2 Deficient Platelets

To investigate the molecular basis of platelet activation, we examined the effect of CLEC-2 (platelet ITAM receptor) deficiency in platelets following liver injury. Mice deficient in CLEC-2 (selectively on platelets, using a PF4 cre system) were studied using either the carbon tetrachloride or acetaminophen (paracetamol) models of acute murine hepatitis.

Importantly, homozygous loss of CLEC-2 in these mice does not give rise to the bleeding diathesis seen with traditional platelet inhibitors, which can often be fatal.

Wild-type (WT) or platelet CLEC-2 deficient mice were injected intraperitoneally with carbon tetrachloride or acetaminophen (paracetamol) and sacrificed ether 24, 48 or 72 hours after injection. Although the initial level of liver injury was similar for both WT and CLEC-2 deficient mice, we found that serum alanine transaminase levels (ALT), a well-established marker of hepatic injury, were markedly reduced in CLEC-2 deficient mice compared to WT mice 48 and 72 hours after carbon tetrachloride injection. Reduced serum ALT levels were also observed in CLEC-2 deficient mice compared to WT mice 24 and 48 hours after paracetamol injection (FIG. 1A).

Following sacrifice, liver tissue was isolated from the carbon tetrachloride or acetaminophen treated WT and CLEC-2-deficient mice. The tissue was paraffin embedded and hematoxylin-eosin stained (FIG. 1B). We found that liver sections from CLEC-2 deficient mice, following injection with either carbon tetrachloride or acetaminophen, had decreased liver injury as evidenced by areas of necrosis (FIG. 1B), compared to WT mice. This suggests that mice with CLEC-2 deficient platelets exhibit improved liver-recovery after a toxic liver injury in comparison to WT mice.

### CLEC1b fl/fl PF4cre Mice Exhibit Greater Hepatic Neutrophil Recruitment than Wild Type Animals after CCL4 Injection

We next sought to study the effect of platelet CLEC-2 deficiency (CLEC1b fl/fl PF4cre mice) upon neutrophil recruitment following liver injury.

Livers from carbon tetrachloride or paracetamol-injured WT and CLEC-2 deficient (selectively in platelets) mice were isolated at 24, 48 or 72 hours post injection, and the number of neutrophils per gram of liver tissue was quantified. Our flow cytometry gating strategy (CD3−, CD45+, GR-1+, CD11b+) for identifying neutrophils is shown in (FIG. 2A). Neutrophil numbers were significantly increased in CLEC-2 deficient mice compared to WT mice at 24 and 48 hours post injection (FIG. 2B). At 72 hours post injection neutrophil numbers were comparable between WT and CLEC-2 deficient mice (FIG. 2B). Microscopy of liver sections (FIG. 2C) also confirmed the increased infiltration of neutrophils into the liver of CLEC-2 deficient mice compared to WT mice post injection.

### CLEC-2 Deficient Platelets Interact with Kupffer Cells and Enhance TNF-alpha Production, thus Increasing Neutrophil Recruitment in CLEC1b fl/fl PF4cre (KO) Mice

We next sought to determine why CLEC-2 deficient mice demonstrated increased neutrophil recruitment post-liver injury.

Kupffer cells isolated from WT mouse livers were plated in a tissue culture well, treated with lipopolysaccharide (LPS) and incubated with either CLEC2 deficient platelets or WT platelets. The production of TNF-alpha by the Kupffer cells was then measured using a capture ELISA. Interestingly, higher levels of TNF-alpha were produced by the Kupffer cells incubated with CLEC2 deficient platelets than from the Kupffer cells incubated with WT platelets (FIG. 3A). This trend was also observed in vivo (FIG. 3C); serum TNF-alpha levels from CLEC1b fl/fl PF4cre (KO) mice were significantly higher than serum TNF-alpha levels from WT mice at 24 hours after carbon tetrachloride injection.

The interaction between Kupffer cells and the platelets was explored in more detail in vitro (FIG. 3B) and in vivo (FIG. 3F). The incubation of isolated Kupffer cells with either CLEC2 deficient or WT platelets (FIG. 3B) revealed that CLEC2 deficient platelets interact in substantially greater numbers with Kupffer cells than WT platelets. This trend was also observed in vivo (FIG. 3F) in frozen mouse liver tissue from CLEC1b fl/fl PF4cre (KO) mice.

To establish if the increased TNF-alpha levels was responsible for the increased neutrophil recruitment in the CLEC1b fl/fl PF4cre (KO) mice, CLEC1b fl/fl PF4cre mice were pre-treated with an anti-TNF-alpha monoclonal antibody (Etanercept) before carbon tetrachloride injection (KO+AB). Mice were sacrificed 48 hours after the carbon tetrachloride injection, and the number of neutrophils per gram of liver tissue calculated (FIG. 3D). The CLEC1b fl/fl PF4cre mice pre-treated with Etanercept had neutrophil numbers which were substantially reduced compared to CLEC1b fl/fl PF4cre mice that were not pre-treated with antibody. The neutrophil numbers of the pre-treated mice were comparable to WT control (FIG. 3D). This suggests that the increased TNF-alpha production in CLEC1b fl/fl PF4cre mice may contribute to increased neutrophil recruitment in the liver. Serum ALT levels from the groups in (FIG. 3D) were also measured at the same time point (48 hours post carbon tetrachloride). Interestingly, the serum ALT levels of the TNF-antibody pre-treated mice were considerably higher than the corresponding group of CLEC-2 deficient mice not given antibody, indicating that the increased TNF-alpha and thus increased neutrophil recruitment is important for liver recovery.

### Podoplanin is Upregulated During Toxic Injury by Macrophages in Human and Mouse Livers

Podoplanin is the only known naturally occurring CLEC-2 ligand. We therefore decided to study the expression of podoplanin in mice and humans following liver injury.

Fodoplanin was upregulated on cells within the inflammatory infiltrate during acetaminophen-induced human liver injury (FIG. 4A). This upregulation was not observed in uninjured liver. Similar upregulation of podoplanin was also observed in injured mouse liver (FIG. 4C).

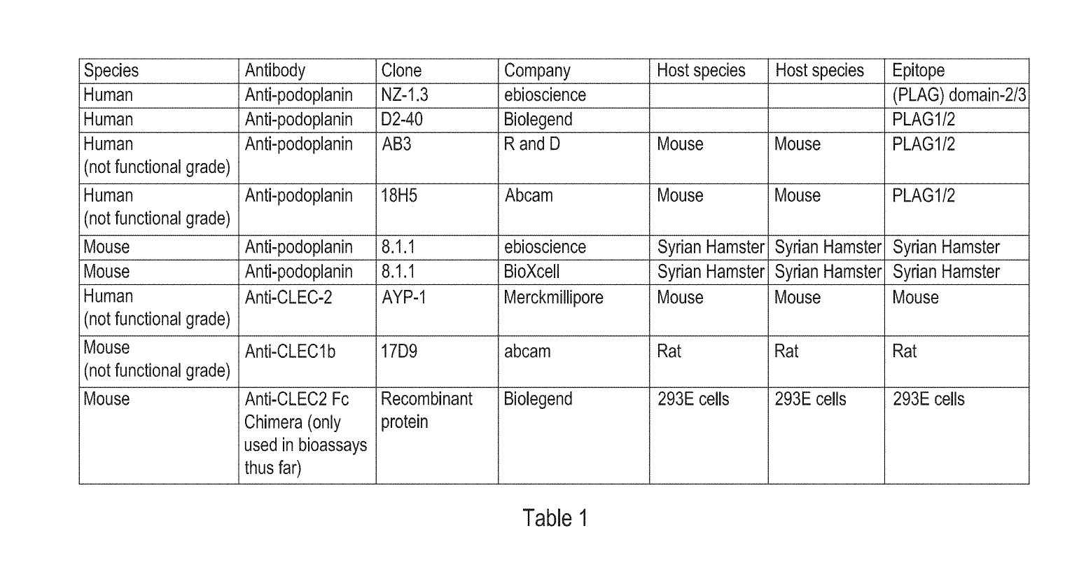

Further analysis of the human cells expressing podoplanin following acetaminophen liver injury found that a significant proportion of these cells are hepatic macrophages or Kupffer cells (FIG. 48).

### Podoplanin Deficient Mice Exhibit Enhanced Neutrophil Recruitment and Reduced Liver Failure Compared to Wild-Type Mice

We next explored the effect of podoplanin deficiency on neutrophil recruitment. WT or Vav-1 cre (podoplanin-deficient mice) were injected with carbon tetrachloride and sacrificed 48 hours after injection. The number of neutrophils per gram of liver tissue was then calculated. Neutrophil recruitment was increased in podoplanin-deficient Vav-1 cre mice compared to WT mice (FIG. 5A). In addition, liver failure, as assessed by ALT serum levels was reduced in podoplanin-deficient vav-1 cre mice compared to WT mice 48 hours post carbon tetrachloride injection (FIG. 5B). Hematoxylin-eosin staining of podoplanin deficient and WT liver sections post injection confirmed that liver failure was reduced in the podoplanin deficient mice compared to WT controls (FIG. 5C). Thus, podoplanin deficiency leads to enhanced neutrophil recruitment and reduced liver failure.

### A Selective Podoplanin Function-Blocking Antibody Reduced Liver Injury by Enhancing Neutrophil Recruitment After Carbon Tetrachloride Induced Liver Injury

We next tested the effect of a selective podoplanin function-blocking antibody on the treatment and/or prophylaxis of liver injury.

WT mice were treated with an intravenous podoplanin blocking antibody (anti-podoplanin) prior to carbon tetrachloride injection. Mice were sacrificed 72 hours after carbon tetrachloride injection. Serum ALT levels were measured at the time of sacrifice. We found that serum ALT levels were significantly reduced in antibody-treated groups compared to WT groups (FIG. 6A).

The number of neutrophils per gram of liver tissue was also calculated from liver tissue harvested at the time of sacrifice. In accordance with the results from podoplanin deficient mice, we observed increased neutrophil numbers in the liver tissue of mice pre-treated with the anti-podoplanin antibody, compared to WT controls (FIG. 6B).

The reduced liver injury and increased neutrophil recruitment suggested by FIGS. 6A and B were confirmed by the microscopic analysis of liver tissue sections from control or anti-podoplanin treated mice (FIG. 6C). Sections were stained with a neutrophil elastase antibody by a DAB stain, and matched serial sections were stained using Haematoxylin and Eosin. FIG. 6C shows increased numbers of brown stained, elastase-positive neutrophils in antibody treated animals (top panels), and that these livers also exhibited less evidence of tissue injury and necrosis when stained using haematoxylin and eosin (FIG. 6C bottom panels).

### Discussion

Our data show that hepatic necroinflarnmation post CCL4 (carbon tetrachloride) or acetaminophen injection is markedly reduced in mice with CLEC-2 deficient platelets. These mice exhibit increased neutrophil recruitment in the liver upon injury. We have data suggesting that this increased neutrophil recruitment is caused by enhanced TNF-alpha production from Kupffer cells in platelet CLEC-2 deficient mice. Our studies have also shown that CLEC-2 deficient platelets undergo increased interactions with Kupffer cells in comparison to WT platelets. It is possible that this increased interaction leads to the upregulation of TNF-alpha expression and/or secretion from the Kupffer cells.

We have also shown that macrophages (F480⁺CD11b⁺) in the inflamed liver up-regulate the only known naturally occurring CLEC-2 ligand, podoplanin. Podoplanin upregulation was observed in both human and mouse acutely inflamed livers. Similarly to CLEC-2 deficient mice (platelet only), podoplanin-deficient mice exhibited enhanced neutrophil recruitment and reduced liver failure, post carbon tetrachloride injection, in comparison to WT mice.

We have further demonstrated that abrogating the platelet based CLEC-2 signal (PF4 Cre mice) or using a function blocking Podoplanin antibody in mouse models of acute hepatic inflammation results in increased neutrophil recruitment to the liver and reduced liver failure.

These findings together indicate that platelets and specifically the CLEC-2 podoplanin axis (pathway shown in FIG. 7) play an important role in acute inflammatory liver disease and thus present an exciting avenue for potential prophylactic and therapeutic treatments for acute liver injury.

1. A method for the treatment or prophylaxis of acute liver failure in a subject, the method comprising the administration of an agent that inhibits an interaction of podoplanin with CLEC-2, or inhibits the activity of Src and/or Syk family kinases to said subject.

2. The method according to claim 1, wherein the agent specifically binds to podoplanin.

3. The method according to claim 1, wherein the agent specifically binds to CLEC-2.

4. The method according to claim 1, wherein the agent comprises an antibody.

5. The method according to claim 4, wherein the antibody is humanised.

6. The method according to claim 1, wherein the acute liver failure is selected from viral-induced liver failure, drug-induced liver failure, alcohol-induced liver failure, autoimmune-induced liver injury, heat-stroke induced liver failure, toxin-induced liver failure, hypoxic hepatitis, or pregnancy induced liver failure.

7. The method according to claim 6, wherein the acute liver failure is alcohol induced or drug induced.

8. The method according to claim 1, wherein the agent is in combination with at least one additional agent, and wherein the at least one additional agent is selected from corticosteroids, N-acetyl cysteine (NAC), or an agent that activates neutrophils.

9. The method according to claim 1, wherein the agent is administered at a timepoint of from 30 seconds to 72 hours post-onset or post-diagnosis of acute liver failure.

10. The method according to claim 1, wherein the agent is administered at a dose of between 0.1 μg/kg of body weight and 1 g/kg of body weight.

11. A composition comprising a therapeutically effective amount of an agent that inhibits an interaction of podoplanin with CLEC-2 or inhibits the activity of Src and/or Syk family kinases, wherein said therapeutically effective amount is sufficient to eliminate, reduce, or prevent acute liver failure.

12. A composition comprising a therapeutically effective amount of a combination of an agent that inhibits an interaction of podoplanin with CLEC2 or inhibits the activity of Src and/or Syk family kinases, and at least one additional agent, wherein the at least one additional agent is selected from corticosteroids, N-acetyl cysteine (NAC), or an agent that activates neutrophils, and wherein said therapeutically effective amount is sufficient to eliminate, reduce, or prevent acute liver failure.

13. The composition according to claim 11, wherein said composition further comprises a pharmaceutically acceptable carrier, diluent or excipient.

14. A method of determining the efficacy of treatment of acute liver failure in a subject using an agent that inhibits an interaction of podoplanin with CLEC-2 or inhibits the activity of Src and/or Syk family kinases, the method comprising:
isolating samples from the subject; and
determining in the samples whether the levels of alanine transaminase (ALT) have decreased after the treatment.

15. The composition according to claim 12, wherein said composition further comprises a pharmaceutically acceptable carrier, diluent, or excipient.
